(19) United States
(12) Patent Application Publication
(10) Pub. No.: US 2026/0154237 A1
(43) Pub. Date: Jun. 4, 2026

(54) System and Method for BAM-Assisted Compression of FASTQ Files

(71) Applicant: Divon Mordechai Lan, Tel Aviv (IL)

(72) Inventor: Divon Mordechai Lan, Tel Aviv (IL)

(21) Appl. No.: 18/964,360

(22) Filed: Nov. 30, 2024

(51) Int. Cl. G06F 16/174; G16B 30/10; G16B 50/50

(52) U.S. Cl. CPC G06F 16/1744; G16B 30/10; G16B 50/50

(57) ABSTRACT

This invention presents a system and method for compressing unaligned genomic sequences in a FASTQ file using alignment information from corresponding in a BAM file and a reference genome. This is useful in cases where both FASTQ and BAM data are available, but the user wishes to store the FASTQ file for the long term while discarding the BAM. Using this invention the BAM file can be used to improve the speed and efficiency of compressing the FASTQ ahead of the BAM file's removal. Key modules include a “populator” for indexing alignments from BAM files, a “consumer” for identifying and compressing matching FASTQ reads, and a “reconstructor” for uncompressing reads. The invention addresses challenges such as differences in read sequences between FASTQ and BAM due to trimming. The invention significantly enhances compression efficiency, reducing storage and transmission requirements while preserving the ability to reconstruct original FASTQ files.

### BACKGROUND

### Field of the Invention

This invention resides in the field of bioinformatics, particularly focusing on the compression of unaligned genomic sequence data, such as that stored in FASTQ or FASTA formats, using information derived from aligned genomic data in BAM or similar formats (for example, SAM or CRAM). This approach aims to address the challenges posed by the substantial storage requirements of genomic data files, which typically range from gigabytes to hundreds of gigabytes in size. By leveraging the relationship between unaligned and aligned data, this invention offers a novel solution for optimizing genomic data storage.

### Description of the Related Art

Genomic sequencing technologies have revolutionized biology and medicine by providing unprecedented insights into the molecular blueprint of life. The sequencing process generates unaligned genomic data in formats like FASTQ or FASTA, which must often be aligned against reference genomes and stored in formats like SAM, BAM or CRAM for downstream analysis. As the genomic field transitions from research to clinical applications, the need for efficient, scalable data management systems has become more pressing than ever.

Effective compression of genomic data is not merely a technical challenge but a critical enabler of progress in research and clinical genomics. As sequencing technologies become more affordable, data storage, processing, and transfer have emerged as bottlenecks. Compression systems that reduce these burdens enable faster collaborations, lower operational costs, and broader access to genomic insights. These systems also pave the way for practical applications in precision medicine, where timely access to data is critical for decision-making. Thus, advancements in data compression directly impact the ability to scale genomic analysis and deliver actionable outcomes.

Genomic data generation is growing at an exponential rate. Sequencing a single human genome typically produces tens of GBs of FASTQ files in addition to tens of GB of aligned BAM files. Large-scale projects like the 100,000 Genomes Project and similar initiatives generate petabytes of data annually, requiring massive storage investments. The cost of storing such data often surpasses the cost of sequencing itself.

Since aligned data is generated from unaligned data (in presence of a reference genome), some users opt to save storage space by discarding the aligned data files after the immediate analysis work is done, and store only the unaligned data for long-term archival purposes, reasoning that the aligned data can re-produced if needed, by aligning the unaligned data once again.

Prior art for compressing unaligned genomic sequences includes tools such as Genozip (Lan, D., et al. (2021) “Genozip: a universal extensible genomic data compressor”, Bioinformatics, 37, 2225-2230; Lan, D., et al. (2022) “Genozip 14—advances in compression of BAM and CRAM files”; Lan, D., et al. (2023) “Deep FASTQ and BAM co-compression in Genozip 15”), PetaGene (D. Greenfield, V. Wittorff and M. Hultner, “The Importance of Data Compression in the Field of Genomics,” in IEEE Pulse, vol. 10, no. 2, pp. 20-23, March-April 2019) and SPRING (Chandak, Shubham, et al. “SPRING: a next-generation compressor for FASTQ data.” Bioinformatics 35.15 (2019 ): 2674-2676), and several others. These tools are typically designed to compress FASTQ files, which in addition to unaligned genomic sequences also include read names and related metadata, and base quality scores. The novelty of this invention is focused on the compression efficiency and speed of the genomic sequences.

One approach to compressing of genomic sequences employed by several prior art tools such as Genozip is known as reference-based compression. In this method, the compressor attempts to find where each unaligned read fits in a reference genome of the organism from which the genomic data is assumed to originate, and if such location is found, it stores the coordinates of the location and any discrepancies between the unaligned read on hand and the segment of the reference data. This reduction of the data from an actual sequence of bases to a description of an alignment against a reference genome is a major contributor to the compression of the unaligned read. However, this process, known in the art as alignment, of finding the location in the reference genome which approximately matches the read and finding the discrepancies between the read and the segment on the reference genome, is expensive in terms of compute resources, and compressors usually seek to strike a balance between the reducing the information content of an alignment (i.e. the number of bits needed to describe it) and the speed of finding one. Aligners within compressors are conceptually similar to aligners that generate aligned files such as BAM files, but with a different objective: the objective of an aligner that generates a BAM file is to find the true location of the read on the biological DNA or RNA molecule from which it originated, and spends considerable compute resources attempting to increase the probability that each alignment is indeed biologically correct. On the other hand, an aligner within a compressor has a much-relaxed objective—merely to find one location on the reference genome that resembles the FASTQ genomic sequence on hand enough to reduce the number of bits needed to describe it.

Another approach to compression of unaligned genomic sequences such as those in a FASTQ file, known as reference-free compression, seeks to find and leverage redundancies within the unaligned genomic data itself. These redundancies exist due to the fact that in common practice, genetic material is typically sequenced many times over, resulting each segment of the genomic material being covered on average, typically, dozens of times. One tool that employs such a strategy is SPRING.

By looking at prior art, multiple innovations have been found in similar domain. For instance, a CN patent 1,066,87966B relates to method and system for data analysis and compression. It provides computer-implemented methods and systems for analyzing data sets (e.g., large data sets output from nucleic acid sequencing technologies). In particular, it provides data analysis that includes computing the BWT of a collection of strings in an incremental character-by-character manner. It also provides a compression lifting strategy that produces a BWT of a rearranged collection of data that is more compressible by a second-stage compression method than a non-rearranged computational analysis.

a U.S. Pat. No. 11,176,103B2 related to representing genomic sequence information using a virtual file system.

a CN patent 1,093,60605B related to a method for compressing genomic sequences by taking advantage of information redundancy between genomic regions.

a US patent application US 20220344005A1 relates to a method to compressed aligned genomic data while concurrently encrypting it.

a US patent application US20210050074A1 relates to compressing unaligned genomic sequences using variable length encoding.

None of the previous inventions and patents, taken either singly or in combination, is seen to describe the instant invention as claimed. Hence, the inventor of the present invention proposes to resolve and surmount existent technical difficulties to eliminate the aforementioned shortcomings of prior art.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

The primary desirable object of the present invention is to provide a novel and improved method to address the compression of unaligned genomic sequences, typically stored within FASTQ or FASTA files.

It is also the objective of the invention to significantly reduce the storage requirements of genomic data without compromising data integrity.

Another object of this invention is to leverage alignment information from BAM files to compress FASTQ files more effectively.

Yet another object of this invention is increasing the speed of finding a suitable alignment for compressing a given unaligned sequence using reference-based compression, specifically by retrieving such an alignment from the BAM rather than the prior art method of computing it by mapping the unaligned read against the reference genome directly.

It is also the objective of the invention to maintain data fidelity during uncompression, ensuring accurate reconstruction of original genomic sequences.

Other aspects, advantages, and novel features of the present invention will become apparent from the detailed description of the invention when considered in conjunction with the accompanying drawings.

This Summary is provided merely for the purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, and Claims.

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The present invention lies within the domain of bioinformatics, specifically addressing the compression of unaligned genomic sequences, typically stored within computer files of the FASTQ or FASTA format. It leverages alignment information from corresponding aligned data files in formats such as SAM, BAM, or CRAM. Unaligned genomic files, often extremely large, pose significant challenges in storage and transmission. The present invention provides a novel approach to compressing unaligned genomic sequences (such as those in FASTQ files) by utilizing alignment information from corresponding alignment files (such as BAM files). By doing so, the invention allows a compressor to save considerable compute resources by bypassing the need to run its own aligner for reads that exist in the BAM file. Furthermore, because aligners used to generate BAM files are typically stricter than those used by compressors and spend considerably more compute resources finding the best alignment, the alignment retrieved from the BAM file is often of better quality than that that would have been produced by the compressor's own aligner, allowing storing a description of the genemic read on hand using less bits, thereby improving the compression efficiency.

Sequencing technologies generate massive datasets, with raw reads stored in formats like FASTQ, while aligned reads are stored in formats like BAM. Some users, desiring to save storage space, opt to store only the unaligned data and discard the aligned data, despite have already spent considerable compute resource generating the latter. This invention improves the compression ratio and speed of compressing of unaligned genomic sequences by using the alignment information in the aligned data, before it is potentially discarded, essentially salvaging some of the compute investment that went into generating the aligned data.

As per its preferred embodiment, the invention consists of three main modules: the populator, which indexes the alignment data in the BAM file; the consumer, which compresses genomic sequences in FASTQ reads by using BAM-derived alignment informaiton; and the reconstructor, which restores original reads during uncompression. These modules are described herein in detail.

The populator module is employed first, as a pre-processing step, to traverse the BAM file and analyze each alignment. If an alignment's FLAG field indicates that it is mapped, non-supplementary and non-secondary, a “bamass” record is created that includes the RNAME, POS and CIGAR of that alignment, and also the length of the sequence and a hash value of the QNAME field (generated with crc64) and of the SEQ field (generated with crc32), as well as a “ext” field used for a linked list. If the reverse-complemented flag is set, the SEQ field is reverse-complemented before the hash function is applied and the operations within the CIGAR string are reversed before storing it, and a rev_comp flag is set within the bamass record. The record is placed in an array in memory, and it is linked to one of the 1,048,576 linked lists—the one linking all the records which share the same 20 least significant bits of the QNAME hash. An index array of length 1,048,576 is maintained to hold the head of each linked list, and all the entries on each particular linked list are linked using the “next” field.

The consumer module is employed when compressing a FASTQ read. The FASTQ read name is hashed similar to the QNAME in the BAM file, and the linked list corresponding to the 20 least significant bits of the hash value is traversed, searching for all the bamass entries on this particular linked list that have the same QNAME hash as the read on hand. For each such entry, we test to see whether the SEQ field's hash matches the hash of the FASTQ sequence. If the BAM sequence is shorter than the FASTQ sequence, it is still possible that the BAM sequence matches a substring of the FASTQ sequence. This happens often, for example, if the software pipeline that processed the FASTQ into the BAM file trimmed the FASTQ read. To detect this case, all the subsequences of the FASTQ sequence of the length matching the BAM's sequence's length are tested for their hash. If such a sequence or subsequence is not located, then this genomic sequence cannot be handled with this method. If such a sequence or subsequence is, in fact, located, then we use the information in the bamass record to represent the FASTQ read in the compressed data stream: we first expand the CIGAR by adding S operations if we detected that trimming occurred to account for this trimming. We then use the CIGAR to traverse the reference genome at the location designated and record any mismatches. An output record consisting of the RNAME, POS, CIGAR, rev_comp flag, a pair (offset, base) for each mismatch and the sequence of bases representing I (insertions) and S (soft clip) operations on the CIGAR is sufficient to represent the FASTQ sequence. The collection of output records is further compressed with the standard zlib compression method before written to the output file.
the reconstructor module is used when uncompressing a compressed FASTQ file, for those genomic sequences within the FASTQ that are represented by an output record. The reconstructor module inspects an output record, traversing the CIGAR and simultaneously traversing the reference genome starting at the location described by RNAME and POS and in the direction implied by the rev_comp flag. Bases are copied from the reference genome (reverse complemented if needed), and insertion, soft clip and mismatching bases from the output record, as programmed in the CIGAR string and mismatch pairs, as commonly done in the art.

In the preferred embodiment, the remainder of the FASTQ file, consisting of the description line of each read, the base quality scores, and genomic sequences which the consumer filed to compress and other data in the FASTQ file are compressed in a separate stream using standard the zlib method. During the uncompress operation, this data is uncompressed using a standard zlib uncompressor and integrated back with the genomic sequences reproduced by the reconstructor, to reproduce the original FASTQ files.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

1. A method for compressing unaligned genomic sequences using alignment information from corresponding aligned data and a reference genome.

2. As per claim 1, the unaligned genomic sequences are contained in data in FASTQ or FASTA format.

3. As per claim 1, the aligned genomic data is represented in SAM, BAM, or CRAM format; and.

4. As per claim 1, a hash-based indexing mechanism is used to correlate FASTQ reads with BAM alignments.

5. As per claim 1, where aligned sequences may be complete or partial sequences of the unaligned sequences, and possibly reverse complimented.

6. As per claim 1, where compressed data is represented such that, it may be uncompressed without use of said corresponding aligned data.

7. A genomic compression apparatus incorporating computer hardware and software to enable compression of a FASTQ file by utilizing alignment information contained in a BAM file and a reference genome.
